(19) United States
(12) Patent Application Publication
(10) Pub. No.: US 2025/0281674 A1
(43) Pub. Date: Sep. 11, 2025

(54) Systems and Methods for Utilizing Partially Digested Cartilage Matrix for Chondral Defects

(71) Applicant: Georgia Tech Research Corporation, Atlanta, GA (US)

(72) Inventors: Nettie Elizabeth Brown, Atlanta, GA (US); Scott J. Hollister, Atlanta, GA (US); Johnna S. Temenoff, Atlanta, GA (US)

(21) Appl. No.: 19/075,212

(22) Filed: Mar. 10, 2025

Related U.S. Application Data
(60) Provisional application No. 63/562,775, filed on Mar. 8, 2024.

(51) Int. Cl. A61L 27/36; A61L 27/38; A61L 27/52; B33Y 80/00

(52) U.S. Cl. CPC A61L 27/3612; A61L 27/3687; A61L 27/3691; A61L 27/3852; A61L 27/52; A61L 2400/06; A61L 2430/06; B33Y 80/00

(57) ABSTRACT

An exemplary embodiment of the present disclosure provides a method of utilizing partially digested cartilage for chondral defects, the method comprising: obtaining cartilage; mincing the cartilage into a plurality of pieces of cartilage; partially digesting the plurality of pieces of cartilage in a digesting mixture to form partially digested cartilage; and encapsulating the partially digested cartilage.

### CROSS-REFERENCE TO RELATED APPLICATIONS

This application claims the benefit of U.S. Provisional Application Ser. No. 63/562,775, filed on 8 Mar. 2024, which is incorporated herein by reference in its entirety as if fully set forth below.

### FIELD OF THE DISCLOSURE

The various embodiments of the present disclosure relate generally to systems and methods for utilizing partially digested cartilage to treat chondral defects.

### BACKGROUND

Current methods for reconstructing cartilaginous facial structures, such as the ear and nose, due to congenital issues, trauma, aesthetic purposes, or tumor removal, are inadequate in meeting patient expectations. Rib cartilage reconstruction is considered the gold standard but involves a painful donor site rib harvest, carries severe risks like pneumothorax, and depends on significant surgical skill, often leading to poor outcomes due to variability. Alloplastic reconstruction is an alternative but comes with significant complications, including extrusion, infection, and fracture.

Tissue-engineered (TE) craniofacial cartilage reconstruction offers a promising alternative to rib and alloplastic reconstruction but faces three major challenges. First, it involves a resorbable scaffold that can replicate complex craniofacial anatomy. Second, this scaffold should effectively deliver sufficient cells to regenerate large volumes of cartilage matrix within the complex anatomy. Third, solutions should be simple enough from a regulatory standpoint to address pressing clinical concerns within a reasonable time and cost.

Many facial surgeries involve the reconstruction of cartilaginous structures, particularly auricular (ear) and nasal cartilage. Facial plastic surgery typically addresses cartilage defects in the nose (ala, side wall, tip), ear, tarsal plate, and orbital wall due to aesthetic purposes, skin cancer resection, congenital deformities (e.g., microtia), and trauma. The number of patients with facial disfigurements from these causes is substantial. For example, in the UK, it affects 569,000 people, or 1 in 111 individuals, which translates to approximately 3 million people in the US. Disfigurement from facial cartilage anomalies significantly impacts hearing, breathing, and psychosocial well-being. Procedures involving significant cartilage volumes include rhinoplasty, the most commonly performed facial plastic surgery, with nearly 200,000 rhinoplasties involving cartilage grafts performed yearly in the US.

Facial cartilage repair presents numerous challenges to reconstructive surgeons. For example, anatomical defects, especially of the ear and nose, are geometrically complex. A significant volume of cartilage tissue is needed to reconstruct defects. Reconstruction should be precise and accurate, as shortcomings in aesthetic reconstruction affect not only function but also psychosocial well-being. Reconstructive materials should have proper stiffness, as skin breakdown and extrusion of reconstructive materials are significant complications in facial cartilage surgery, affecting up to 15% of all auricular reconstructions

Autologous cartilage grafts are the clinical gold standard for facial cartilage reconstruction. Autologous grafts have proper stiffness to avoid skin breakdown and autologous tissue remodels and integrates with host tissue. However, autologous cartilage harvesting has many downsides. First, autologous grafts typically must be reshaped in the operating room (OR) to fit complex geometry, resulting in tremendous variability of outcomes based on surgeon skill. Second, there can be limited material to harvest, restricting options for large defects and revisions. Third, costochondral cartilage rib grafts are the most widely harvested, which cause significant patient pain, carry risks including pneumothorax, and often warp over time.

Autologous grafting limitations have led to the use of synthetic implants for facial cartilage reconstruction, most notably permanent high-density polyethylene (HDPE) Medpor implants. Advantages of synthetic implants include elimination of tissue harvesting with associated complications, reduced number of surgeries, pre-fabricated shapes that eliminate the need for in OR shaping, and no material limitations. However, the stiff synthetic implants have high rates of skin breakdown and implant extrusion compared to autologous grafts. For this reason, permanent synthetic implants like Medpor are used much less often than autologous grafts for facial cartilage reconstruction.

Tissue engineering (TE) has been proposed for 25 years as an alternative to autologous grafts and synthetic implants for facial cartilage reconstruction. TE offers pre-fabricated shapes like synthetic implants with limited patient tissue harvest, but with the advantage of tissue integration and appropriate stiffness of autologous grafts. Despite the promise, TE clinical facial cartilage reconstruction has been reported in only two studies, auricular reconstruction in microtia patients in China and nasal reconstruction following skin cancer resection in Switzerland. Indeed, only five studies have reported auricular reconstruction using high fidelity auricular scaffolds with cartilage cell/tissue in immunocompetent animals (rabbit, pig and sheep).

The question is why, after 25 years and despite the promise of TE, cartilage autografts continue to be the gold standard instead of TE. Part of the reason is the scientific challenges of adequate cell sources, efficient cell delivery and scaffold fabrication to replicate complex facial cartilage anatomy. These challenges can potentially be addressed by combining chondrocytes with stem cells, using engineered hydrogels for efficient cell delivery, and 3D printing scaffolds to replicate facial cartilage anatomy.

However, the failure of TE as a viable therapy for facial cartilage reconstruction goes beyond solving scientific challenges. TE therapies must achieve regulatory approval and economic viability to become the clinical gold standard. TE approaches using harvested cells expanded outside the OR are regulated by the FDA as class III combination products, requiring two clinical trials (with ˜100's of patients), 6-8 years and $10-100 million. To date, no clinical trials for nasal reconstruction and only one for auricular reconstruction enrolling just two patients (Aurinova, closed May 18, 2023) are documented on clinicaltrials.gov. This paucity of clinical trials demonstrates the significant regulatory hurdles using traditional cell expansion for facial cartilage TE.

Finally, clinical adoption of TE therapies is critical to replacing autologous grafting as a clinical gold standard. Regulatory approval is of course a prerequisite to clinical adoption. Economic viability is also required for clinical adoption. There is no TE facial cartilage product for comparison. An analogy is TE articular cartilage repair, where the effectiveness of the only FDA approved TE treatment Matrix-induced Autologous Chondrocyte Implantation (MACI) was compared to cartilage allograft for patellar cartilage lesion repair. MACI for this condition costs $83,073/case while particulated juvenile allograft cartilage (PJAC) cost $52,683/case. Both treatments were considered equally effective, but PJAC was deemed more cost effective than MACI and required one surgery versus MACI's two. Other reports give MACI cost at $40,000/case with allograft and autograft at $14,000 and $11,000, respectively. No matter the source, TE MACI using laboratory cell expansion costs between 1.6× and 3.6× cartilage autograft and allograft. If TE treatments are not superior to grafts but cost on average twice as much, they will not be widely adopted for clinical use. As a comparison, rhinoplasty using costochondral rib graft averages $8,342 (which increases to $21,099 with pneumothorax complications). Taking articular cartilage as a benchmark, facial cartilage TE should cost no more than $13K to $30K/case.

Accordingly, there is a need for improved techniques for addressing chondral defects that overcome one or more of the disadvantages discussed above.

### BRIEF SUMMARY

An exemplary embodiment of the present disclosure provides a method of utilizing partially digested cartilage for chondral defects, the method comprising: obtaining cartilage; mincing the cartilage into a plurality of pieces of cartilage; partially digesting the plurality of pieces of cartilage in a digesting mixture to form partially digested cartilage; and encapsulating the partially digested cartilage.

In any of the embodiments disclosed herein, obtaining cartilage can comprise harvesting the cartilage from a subject (e.g., a human patient, either inside or outside the operating room).

In any of the embodiments disclosed herein, the method can further comprise cleaning the harvested cartilage.

In any of the embodiments disclosed herein, the partially digested cartilage can comprise a plurality of chondrocytes with surrounding extracellular matrix.

In any of the embodiments disclosed herein, the plurality of pieces of cartilage can have at least one of an average volume from about 0.1 mm³ to about 5 mm³.

In any of the embodiments disclosed herein, the partially digested cartilage can be encapsulated in a polymer.

In any of the embodiments disclosed herein, the polymer can be a biodegradable polymer.

In any of the embodiments disclosed herein, the polymer can comprise poly(ethylene glycol) diacrylate (PEGDA).

In any of the embodiments disclosed herein, the polymer can be in the form of a hydrogel.

In any of the embodiments disclosed herein, the hydrogel can be incorporated into a 3D printed scaffold. In some embodiments, the scaffold can be designed based on a patient/subject anatomy.

In any of the embodiments disclosed herein, the hydrogel can comprise an organic matrix.

In any of the embodiments disclosed herein, the method can further comprise employing the encapsulated partially digested cartilage into a biodelivery vehicle.

In any of the embodiments disclosed herein, the biodelivery vehicle can comprise one or more selected from the group consisting of: cells, proteins, genes, peptides, and drugs.

In any of the embodiments disclosed herein, the digesting mixture can be in the form of a solution.

In any of the embodiments disclosed herein, the digesting mixture can comprise a digesting enzyme.

In any of the embodiments disclosed herein, the digesting enzyme can be selected from the group consisting of: collagenases, aggrecanases, cathepsins, dispase, pronase, trypsin, hyaluronidase, elastase, plasmin, stromelysins, serine proteases, gelatinases, chymotrypsin, and pepsin.

In any of the embodiments disclosed herein, the digesting mixture can comprise one or more selected from the group consisting of: amphotecerin B, penicillin/streptomycin, non-essential amino acids, L-glutamine, and digesting enzyme.

In any of the embodiments disclosed herein, the digesting mixture can comprise amphotecerin B, penicillin/streptomycin, non-essential amino acids, L-glutamine, and digesting enzyme.

In any of the embodiments disclosed herein, the digesting mixture can be at a concentration of from about 1-100 mg, of minced cartilage per mL of mixture.

In any of the embodiments disclosed herein, partially digesting the plurality of pieces of cartilage in a digesting mixture can comprises placing the digestion mixture in an incubator for a period of time.

In any of the embodiments disclosed herein, the period of time is no more than 24 hours.

Another embodiment of the present disclosure provides a method of utilizing partially digested cartilage for chondral defects, the method comprising: partially digesting a plurality of pieces of cartilage in a digestion mixture comprising at least one enzyme to form a partially digested cartilage; and inserting the partially digested cartilage into a body of a subject proximate a chondral defect.

In any of the embodiments disclosed herein, the method can further comprise, prior to partially digesting a plurality of pieces of cartilage, obtaining a piece of cartilage from the subject and mincing the piece of cartilage to form the plurality of pieces of cartilage.

In any of the embodiments disclosed herein, the method can further comprise prior to inserting the partially digested cartilage into a body of a subject, encapsulating the partially digested cartilage in a hydrogel.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides flow chart of a method of utilizing partially digested cartilage for chondral defects, in accordance with an exemplary embodiment of the present disclosure.

FIG. 2 provides flow chart of a method of utilizing partially digested cartilage for chondral defects, in accordance with an exemplary embodiment of the present disclosure.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

The present disclosure aims to address the scientific challenges of cartilage reconstruction, such as sourcing cells, efficient cell delivery, and creating scaffolds that match complex anatomy. It introduces 3D printed, patient-specific scaffolds combined with heparin-based PEGDA hydrogels of varying sulfation levels, designed for assembly within the operating room (OR). These hydrogels can deliver minced, partially digested cartilage along with adipose cells directly in the OR, eliminating the need for external laboratory cell expansion. This approach is intended to streamline regulatory approval, ideally following a class II 510k path rather than the more complex class III PMA path for cell expansion therapies. The disclosed techniques utilize OR-ready composite scaffolds and cell harvesting methods to enhance the formation of homogeneous cartilage constructs and support the in vivo production of anatomically-shaped, large-volume cartilage tissue.

The disclosure also presents innovative strategies for using partially digested cartilage tissue during surgery for cartilage defects. These strategies may include patient-sourced tissue, targeted cartilage zone digestion, hydrogel encapsulation, microparticle encapsulation, integration with 3D printed scaffolds, composite materials, and growth factor-doped delivery systems.

The partially digested cartilage matrix can be particularly useful in cartilage repair procedures across various sites, including joints, nasal, auricular, and articular cartilage. Its application in the OR enables quicker surgical procedures and reduces the need for in vitro cell expansion. Additionally, the presence of native extracellular matrix proteins in the partially digested matrix minimizes the requirement for exogenous growth factors.

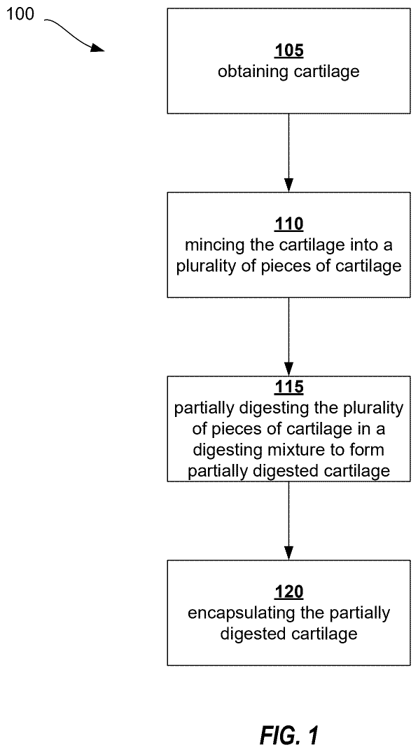

As shown in FIG. 1, an exemplary embodiment of the present disclosure provides a method 100 of utilizing partially digested cartilage for chondral defects. The method can begin at step 105 with obtaining a piece of cartilage. In some embodiments, this can be done by harvesting a piece of cartilage from a subject, e.g., rib area, as is known in the art. In some embodiments, the subject can be a subject with the chondral defect. In some embodiments, however, the cartilage can be a piece of cartilage harvested from another subject, or a piece of artificial cartilage grown in a lab. In some embodiments, in can be desirable to wash/clean the cartilage prior to taking further actions (e.g., mincing). Many substances known the art can be utilized to wash/clean the cartilage, including, but not limited to, saline solutions (e.g., HBSS) and the like.

The method can continue at step 110 with mincing the cartilage into a plurality of pieces. As used herein, the term “mincing” should be broadly construed to include many different means of separating a larger piece of cartilage into a plurality of smaller pieces, e.g., cutting. The plurality of pieces can be many different sizes and/or shapes in accordance with various embodiments of the present disclosure. In some embodiments, each of the plurality of pieces can be the same size, and in some embodiments, the plurality of pieces can be in multiple sizes. In some embodiments, the plurality of pieces of cartilage can be minced such that they have at least one of an average length or an average width from about 0.1 mm to about 3 mm, though the disclosure is not so limited. In some embodiments, the pieces can have an average volume of about 0.1-3 mm³, about 0.1-1 mm³, or about 1-1 mm³.

The method can continue at step 115 with partially digesting the plurality of pieces of cartilage in a digesting mixture to form partially digested cartilage. The digestion mixture can be many different digestions mixtures. In some embodiments, the digestion mixture can be in the form of a solution. In some embodiments, the digestion mixture can comprise at least one digestion enzyme. The digestion enzyme can be many digestion enzymes known in the art, including, but not limited to, collagenases, aggrecanases, cathepsins, dispase, pronase, trypsin, hyaluronidase, elastase, plasmin, stromelysins, serine proteases, gelatinases, chymotrypsin, and pepsin, and the like. The concentration of the digestion enzyme in the digestion mixture can vary in accordance with many embodiments of the present disclosure, e.g., 0.1%-5%, 1%, 1-2.4%, 2.4-7%, and the like, though the disclosure is not limited to any particular concentration. The digestion mixture can also comprise many other ingredients, including, but not limited to, a nutrient mixture (e.g., Ham's F-12), amphotecerin B, penicillin/streptomycin, non-essential amino acids, L-glutamine, digestion enzyme, and the like. An exemplary digestion mixture is in a solution and comprises Ham's F-12, 1% amphotecerin B, 1% penicillin/streptomycin, 1% non-essential amino acids, 0.1% L-glutamine, and 1 mg/mL digestion enzyme.

The plurality of pieces of cartilage can be partially digested in the digestion mixture over a period of time. In some embodiments, the digestion can take place in an incubator. As those skilled in the art will appreciate, the period of time can vary in accordance with various parameters, including, but not limited to, the size of the cartilage pieces, the concentration of the digestion enzyme(s) in the digestion mixture, the concentration of the cartilage in the digestion mixture, temperature/airflow inside the incubator, and the like. In some embodiments, the period of time may be less than four hours, though the disclosure is not so limited. In some embodiments, the period of time is from about 30 minutes to about four hours.

As discussed above, the concentration of the cartilage in the digestion mixture is one parameter than can alter the digestion process. In some embodiments, the digesting mixture can be at a concentration of from about 5-15 mg, 8-12 mg, or about 10 mg-100 mg, of minced cartilage per mL of mixture, though the disclosure is not so limited.

At the end of the digestion process, the partially digested cartilage can comprise a plurality of chondrocytes (e.g., cartilage cells) still at least partially surrounded by extracellular matrix (ECM). As described below, the presence of the ECM can provide benefits over conventional techniques in which cartilage is fully digested.

The method can continue at step 120 with encapsulating the partially digested cartilage. The partially digested cartilage can be encapsulated in numerous materials for delivery. Polymeric materials could be utilized for none, some but not all, or all components, which may include but are not limited to materials such as poly(ethylene glycol) diacrylate (PEGDA). Some embodiments may comprise composite materials in order to match the mechanical behavior of specific tissues, for example by layering an organic matrix into a polymer.

In some embodiments, the partially digested matrix can be formed of a material comprising a polymer, such as a biocompatible or biomedically acceptable polymer that may be biodegradable or non-biodegradable. The term “biodegradable” as used herein means that the implant comprising the polymer is slowly dissolved or disintegrated under physiological conditions in the human or other animal subject for a certain time and at some point only its degradation products are present in the body in a dissolved or comminuted form. At this point, solid components or fragments of the splinting either do not exist anymore or are so small as to be non-harmful or transported away by the subject's circulatory system. The degradation products can be substantially harmless in physiological terms and lead to molecules that either occur naturally in the human or other animal subject or can be excreted by the human or other animal subject.

In various embodiments, the partially digested matrix of the present disclosure comprises a polymer, such as a biodegradable polymer. Biodegradable polymers include polycaprolactone, polysebacic acid, poly(octaindiolcitrate), polydioxanone, polygluconate, poly(lactic acid) polyethylene oxide copolymer, modified cellulose, polyhydroxybutyrate, polyamino acids, polyphosphate ester, polyvalerolactone, poly-6-decalactone, polylactonic acid, polyglycolic acid, polylactides, polyglycolides, copolymers of the polylactides and polyglycolides, polye-caprolactone, polyhydroxybutyric acid, polyhydroxybutyrates, polyhydroxyvalerates, polyhydroxybutyrate-co-valerate, poly(1,4-dioxane-2,3one), poly(1,3-dioxane-2-one), poly-para-dioxanone, polyanhydrides, polymaleic acid anhydrides, polyhydroxy methacrylates, fibrin, polycyanoacrylate, polycaprolactone dimethylacrylates, poly-3-maleic acid, polycaprolactone butyl acrylates, multiblock polymers from oligocaprolactonediols and oligodioxanonediols, polyether ester multiblock polymers from PEG and poly(butlylene terephthalates), polypivotolactones, polyglycolic acid trimethyl carbonates, polycaprolactone glycolides, poly(methyl glutamate), poly (DTH-iminocarbonate), poly(DTE-co-DT-carbonate), poly(bisphenol A-iminocarbonate), polyorthoesters, polyglycolic acid trimethyl carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-(carboxyphenoxy) propane], polyhydroxy pentanoic acid, polyanhydrides, polyethylene oxide propylene oxide, and combinations thereof. In various embodiments, a preferred biodegradable polymer that forms the partially digested matrix system comprises, or consists of, polycaprolactone. In various embodiments, the partially digested matrix system of the present technology comprises a natural polymers including but not limited to cellulose, carrageenan, hyaluronic acid, polysaccharides (alginate, starch, agarose), chitosan, fibrin, and proteins (gelatin, collagen), etc.

In some embodiments, the partially digested matrix comprising the biodegradable polymer allows the cartilage defect to heal naturally and then biodegrade or resorb in the subject. Having the partially digested matrix system biodegrade can eliminate the need for a second surgery for the removal of the implanted system and furthermore may not inhibit cartilage regrowth in adults or growth in children. In some embodiments, the partially digested matrix system can be designed to have a degradation time that coincides with the healing time of the defect in the subject. “Degradation time” refers to the time for the implantable partially digested matrix system to substantially and fully dissolve, disintegrate, or resorb. Depending upon the subject and the time needed for recuperation and regeneration of the cartilage, the degradation time may be about 3 weeks to about 60 months (5 years), or about 2 months to about 40 months (3.33 years), or about 6 months to about 36 months (3 years), or about 12 months to about 24 months (2 years).

In some embodiments the partially digested cartilage can be encapsulated in a hydrogel, which can comprise PEGDA. An exemplary description of how this can be implemented is as follows. The development of PEGDA for use as a material component of a partially digested cartilage delivery system follows a workflow, where PEG is diacrylated for use. The resultant polymer can then be crosslinked with the partially digested material. In brief, PEG (3.4 kDa) can be reacted with acryloyl chloride (AcCl) in an 8:1 AcCl to PEG molar ratio and dissolved in dichloromethane (DCM). To yield linear PEGDA, a catalyst, triethylamine (TEA), can be added dropwise in a 1:1 TEA to AcCl molar ratio. Potassium chloride can be used to separate PEGDA into an organic phase before being precipitated in diethyl ether. Vacuum filtration can be utilized to separate PEGDA from diethyl ether followed by air drying and purging with nitrogen. 10 wt. % dithiothreitol (DTT) can be crosslinked to PEGDA to initiate hydrolytic degradation. PEGDA-DTT mixture can then be mixed initiators for thermal crosslinking like ammonium persulfate (APS) (0.018M) followed by tetramethylethylenediamine (TEMED) as a catalyst (0.018M). Partially digested cartilage can be mixed into the system before crosslinking to provide a homogenous matrix of chondrocytes and extracellular matrix. After 20 minutes, the partially digested hydrogels can be formed. While this method can be utilized to manufacture some embodiments, others may utilize other materials including but not limited to PEGDA with different ultraviolet (UV) crosslinking systems using photoinitiators like Igracure 2959 (1-[4[(2-hydroxyethoxy)-phenyl]-2-hydroxy-2-methyl-1-propane-1-one_and lithium phenyl-2,4,6-trimethylbenzoylphosphinate (LAP).

Some embodiments also perform bioactive delivery, which may include but is not limited to cells, proteins, genes, peptides, and drugs. For example, in some embodiments, the partially digested cartilage matrix may have their surface and/or bulk doped with any number of molecules for any number of purposes, including but limited to the delivery of drugs, which may or may not be released, which may occur over any timeline. Another example of bioactive delivery is the delivery of cells, possibly cells harvested from the patient, which are incorporated into the device, perhaps via cell printing or ingrowth during an in vitro incubation period, or via any other method.

In some embodiments, the partially digested matrix system of the present technology can further comprise one or more bioactive materials. Depending on such factors as the bioactive material, the structure of the partially digested matrix system, and the intended use of the partially digested matrix system, the bioactive material may be coated on a surface of the partially digested matrix system, coated or otherwise infused in the pores or openings of the partially digested matrix system, or mixed or compounded within the polymeric material of the partially digested matrix system. Bioactive materials can include any natural, recombinant or synthetic compound or composition that provides a local or systemic therapeutic benefit. In various embodiments, the bioactive material promotes healing and growth of a damaged cartilage or a degenerating cartilage. Bioactive materials among those useful herein include cell adhesion factors, isolated tissue materials, growth factors, peptides and other cytokines and hormones, pharmaceutical actives, and combinations thereof. Cell adhesion factors include, for example, the RGD (Arg-Gly-Asp) sequence or the IKVAV (Ile-Lys-Val-Ala-Val) sequence. Isolated tissue materials include, for example, whole blood and blood fractions (such as red blood cells, white blood cells, platelet-rich plasma, and platelet-poor plasma), collagen, fibrin, acellularized dermis, isolated cells and cultured cells (such as hemopoietic stem cells, mesenchymal stem cells, endothelial progenitor cells, fibroblasts, reticulocytes, adipose cells, and endothelial cells). Growth factors and cytokines useful herein include transforming growth factor-beta (TGF-I3), including the five different subtypes (TGF-I3 1-5); bone morphogenetic factors (BMPs, such as BMP-2, BMP-2a, BMP-4, BMP-5, BMP-6, BMP-7 and BMP-8); platelet-derived growth factors (PDGFs); insulin-like growth factors (e.g., IGF I and II); fibroblast growth factors (FGFs), vascular endothelial growth factor (VEGF), epidermal growth factor (EGF) and combinations thereof. Examples of pharmaceutical actives include antimicrobials, antifungals, chemotherapeutic agents, and anti-inflammatories. Examples of antimicrobials include triclosan, sulfonamides, furans, macrolides, quinolones, tetracyclines, vancomycin, cephalosporins, rifampins, aminoglycosides (such as tobramycin and gentamicin), and mixtures thereof.

In some embodiments, the hydrogel can be incorporated into a 3D printed scaffold. The surgical delivery method may play a large role in the design. For cartilage resurfacing or small defects, arthroscopic delivery methods using hydrogels or microparticles may be ideal. For larger segments of cartilage defects, especially those that need structural support, delivery systems with stiffer scaffolds like 3D printed devices or porous scaffolds may be sufficient for application.

Some embodiments of the present disclosure can utilize PCL for a scaffolding material. Based upon the mechanical properties of PCL, it may serve especially well as a material used a frame subassembly. One potential 3D printing method, SLS, can be used to manufacture the PCL components utilized in some such embodiments within this family. For this printing method, the PCL can be synthesized then milled into a powder using any number of methods which may include but are not limited to hammer milling, jet milling, and other processes which lend themselves to polymers like PCL, particularly those processes which can be conducted at cryogenic temperatures. The size of PCL powder is a factor which may likely affect printing, and parameters not just of size range but also average and standard deviation are data which will likely be considered and customized. The PCL powder may be mixed with one or more other materials prior to printing, for example to improve the behavior of the powder during printing or of the printed component. Other factors will likely be considered, such as the need to control the presence of water in the powder and in the air during printing, which may for example require desiccating the powder prior to printing and humidity control in the 3D printing environment. Pre-processing may not be required, and if it is, it may require any number of steps for any number of purposes, including but not limited to mixing with additives and desiccating as described herein. The SLS printing process has numerous parameters than may potentially be control to ensure optimal printing, which may include but are not limited to processing chamber temperature and humidity, removal chamber temperature, laser intensity, speed, and wavelength, and layer thickness. After printing, there may be post-processing steps, such as sonication in an ethanol bath. Additional processes which may be conducted on the component include sterilization, which could utilize one or more of any number of methods, including but not limited to EtO, EtOH, UV, and NOx. Packaging is another potential consideration, as is quality control. Quality control for such components may include any number of methods to evaluate any number of parameters. For such SLS manufactured PCL components, this may include uCT to evaluate porosity and compression testing to evaluate mechanical strength.

One exemplary protocol which can be used to manufacture such an embodiment is as follows. The PCL is synthesized and subsequently milled (by third parties) to a powder in the range of 50 to 70 um using liquid jet milling at cryogenic temperatures. The PCL powder is then mixed with 4% hydroxylapatite (HA) by weight. The relative humidity (RH) of the powder mixture is then measured and reduced, if necessary, by storing it in a chamber sealed from the surrounding environment with consistent nitrogen flow. The RH must be below 15% and ideally below 10% prior to printing with it. A Formiga P110 (EOS) is used, which is installed in a cleanroom along with a continual powder supply module for the event power is lost temporarily. Before printing, the room, printer, and associated equipment is thoroughly cleaned using 70% ethanol. The dry powder mixture is then used to print one or more components. During the printing process, the printer's processing chamber is held at roughly 54 C, while the removal chamber is held at roughly 43 C.

The 3D printing of the device can be conducted using any number of 3D printing methods. Some components may be manufactured using a different method and then assembled with the 3D printed component(s). Choice of materials and geometry will likely guide the 3D printing method used. Some embodiments of the manufacturing process include but are not limited to extrusion, selective laser sintering (SLS), direct metal laser sintering (DMLS), digital light processing (DLP), fused deposition modeling (FDM), fused filament fabrication (FFF), PolyJet, stereolithography (SLA), multi jet fusion (MJF), electron beam melting (EBM), solid freeform fabrication (SFF), any combination thereof, and any other additive manufacturing technique existing or to be developed in the future. The material, prior to printing, could be in any form, including but not limited to resin, melt, filament, and powder. There may be processing before and/or after printing. In some embodiments this processing comprises thermally curing a resin after printing to improve mechanical strength.

In some embodiments, the 3D printed materials can comprise a number materials and bioinks. Some embodiments of the materials include but are not limited to polyhydroxyalkanoates (PHA), polyvinyl alcohol (PVA), Polyethylene terephthalate (PET) and High impact polystyrene (HIPS), poly(lactic acid), any combination of thereof, and any other biodegradable/resorbable materials developed in the future.

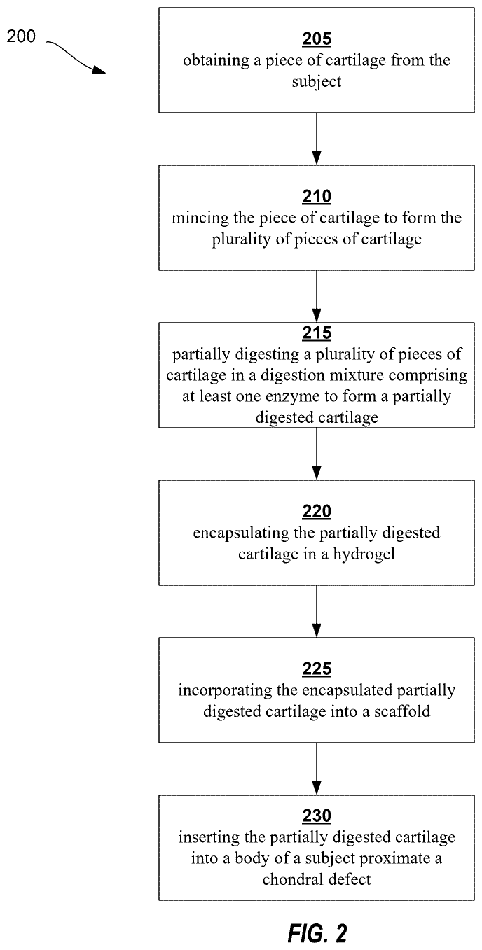

FIG. 2 provides another exemplary method 200 of utilizing partially digested cartilage for chondral defects. At its simplest form, the method can comprise step 215 of partially digesting a plurality of pieces of cartilage in a digestion mixture comprising at least one enzyme to form a partially digested cartilage and step 230 of inserting the partially digested cartilage into a body of a subject proximate a chondral defect. In some embodiments, however, the method can comprise one or more other steps. For example, in some embodiments, the method 200 can further comprise step 205 of obtaining a piece of cartilage from the subject. In some embodiments, the method 200 can further comprise step 210 of mincing the piece of cartilage to form the plurality of pieces of cartilage. In some embodiments, the method 200 can further comprise step 220 of encapsulating the partially digested cartilage in a hydrogel. And in some embodiments, the method 200 can further comprise step 225 of incorporating the encapsulated partially digested cartilage into a scaffold.

### Advantages and Improvements Over Existing Methods, Devices or Materials

Current methods for articular cartilage repair include direct cartilage transplantation methods like mosaic transplantation, osteochondral transplantation, etc., cell therapy strategies like autologous chondrocyte implantation, matrix-induced autologous chondrocyte implantation, etc. and bone marrow stimulation techniques like micro-drilling, micro-fracture, etc. While these strategies are used in the clinic, they can be limited by donor tissue, cell culturing steps, trauma, etc. Surgical reconstruction is the current gold standard of treatment for auricular and nasal cartilage which involves the use of autologous rib (costal) cartilage to rebuild the auricle. However, donor site morbidity, multiple surgery procedures, and pneumothorax are disadvantages to this strategy. The present invention system is a reliable treatment strategy to decrease donor volume needed and cell culture procedures while providing the method to be performed at the time of surgery. This can eliminate the need for multiple surgeries, reducing risk of complications and overall treatment costs.

The unique use of a partially digested cartilage matrix combined with delivery vehicles created numerous advantages over existing methods. Current tissue engineering strategies cited have used autologous minced cartilage in defects and autologous cells with or without biological adhesives like fibrin glue. Both approaches can be valuable for different applications, especially smaller defects (<2 cm²), but our system could be applicable for small and large defects. By encapsulating the partially digested cartilage in delivery vehicles like hydrogels we can fill defects or resurface cartilage defects. One drawback of minced cartilage is the limited potential of chondrocytes to migrate from the dense network of collagen; therefore, in some embodiments of the present disclosure, we partially digest the collagen matrix using digestion enzymes, allowing increased mobility of the chondrocytes. A limitation of autologous chondrocyte implantation is the need to passage the cells in vitro in order to grow the number of cells needed for cartilage tissue regeneration. This method can require a second surgery and chondrocytes tend to dedifferentiate over multiple passages which can lead to fibrotic tissue growth at the defect site. Due to the dedifferentiation potential of chondrocytes, researchers use cell culture media supplemented with growth factors to main chondrocyte phenotype, but this can increase the cost of these procedures. With the partially digested systems disclosed herein, the native extracellular matrix (ECM) proteins and bioactive factors found in native cartilage can remain to maintain the chondrocyte phenotype. Our approach is a gentle digestion system which is beneficial to maintain chondrocyte viability and matrix support. In addition, researchers have previously used decellularized cartilage tissue as scaffolds to promote cartilage regeneration which supports the use of native cartilage ECM due to collagens, proteins, and bioactives for cartilaginous matrix production. Our approach limits the use of out-of-the-operating room procedures like decellularization and lyophilizing to provide a “time of surgery” strategy.

Another benefit of embodiments of the present disclosure over existing technologies is their ability to be combined with numerous materials for delivery. The initial delivery vehicle used was hydrogels. The hydrogels can be crosslinked relatively quickly for application. Due to the Newtonian nature of hydrogels, they can be easily delivered (in a liquid state) through syringes, needles, etc., and inserted into a joint or another cartilage defect site using arthroscopic tools and minimally invasive incisions. In addition, degradable hydrogels can be ideal to allow nutrient diffusion, cell migration, and tissue integration at the defect site to initiate cartilage matrix production. This partially digested cartilage encapsulated hydrogel system being resorbable can allow for regrowth and remodeling of the cartilage tissue from the patient's cells and tissues. In some embodiments, the hydrogels are naturally sourced like collagens, fibrin, alginate, etc. In some embodiments, the hydrogel can be combined with stiffer scaffolds to provide functional support at load-bearing sites. The combination with porous scaffolds, especially 3d printed scaffolds, could provide avenues for patient-specific geometries to fill defects while providing structure to support cartilage ingrowth.

### Commercial Applications (Economic Potential, Direct Uses, Indirect or Potential Uses, Etc.)

Cartilage defects and repair are common, and embodiments of the present disclosure aim to target this market, providing ideal solutions for a uniquely wide range of cartilage applications. Embodiments of the present disclosure can be used to treat any number of diseases which include but are not limited to rheumatoid arthritis at joints, osteoarthritis, microtia, deviated septum, nasal valve collapse, and cartilage damage due to trauma. Embodiments of the present disclosure can be used to repair or replace any number of anatomies which include but are not limited to articular, tracheal, auricular, and nasal cartilage. Thus, different embodiments may have different combinations with delivery systems. Delivery systems encompassing injectable delivery systems like hydrogels or microparticles are likely to be in higher demand than others.

Variations in deployment methods between embodiments can further allow ease of handling for surgeons, otolaryngologists, orthopedic surgeons, clinicians, and other medical professionals. Commercial applications include injectable cartilage defect filler which can be performed arthroscopically and cartilage replacement for larger defects.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method of utilizing partially digested cartilage for chondral defects, the method comprising:
obtaining cartilage;
mincing the cartilage into a plurality of pieces of cartilage;
partially digesting the plurality of pieces of cartilage in a digesting mixture to form partially digested cartilage; and
encapsulating the partially digested cartilage.

2. The method of claim 1, wherein obtaining cartilage comprises harvesting the cartilage from a subject.

3. The method of claim 1, wherein the partially digested cartilage comprises a plurality of one or more chondrocytes with surrounding extracellular matrix.

4. The method of claim 1, wherein the plurality of pieces of cartilage have at least one of an average volume from approximately 0.1 mm³ to about 5 mm³.

5. The method of claim 1, wherein encapsulating the partially digested cartilage comprises encapsulating the partially digested cartilage in a polymer.

6. The method of claim 5, wherein the polymer is a biodegradable polymer

7. The method of claim 5, wherein the polymer is in the form of a hydrogel.

8. The method of claim 7, further comprising incorporating the encapsulated partially digested cartilage into a 3D printed scaffold.

9. The method of claim 1, further comprising employing the encapsulated partially digested cartilage into a biodelivery vehicle selected from the group consisting of: cells, proteins, genes, peptides, and drugs.

10. The method of claim 1, wherein the digesting mixture comprises a digesting enzyme.

11. The method of claim 11, wherein the digesting enzyme is selected from the group consisting of collagenases, aggrecanases, cathepsins, dispase, pronase, trypsin, hyaluronidase, elastase, plasmin, stromelysins, serine proteases, gelatinases, chymotrypsin, and pepsin.

12. The method of claim 1, wherein the digesting mixture comprises amphotecerin B, penicillin/streptomycin, non-essential amino acids, L-glutamine, and digesting enzyme.

13. The method of claim 1, wherein the digesting mixture is at a concentration of from about 1-100 mg of cartilage per mL of mixture.

14. The method of claim 1, wherein partially digesting the plurality of pieces of cartilage in the digesting mixture comprises placing the digestion mixture in an incubator for a period of time of no more than 24 hours.

15. A method of utilizing partially digested cartilage for chondral defects, the method comprising:
partially digesting a plurality of pieces of cartilage in a digestion mixture comprising at least one enzyme to form a partially digested cartilage; and
inserting the partially digested cartilage into a body of a subject proximate a chondral defect.

16. The method of claim 15, wherein the partially digested cartilage comprises a plurality of chondrocytes with surrounding extracellular matrix.

17. The method of claim 15, wherein the digestion mixture is at a concentration of from about 1-100 mg of cartilage per mL of digestion mixture.

18. The method of claim 15, further comprising, prior to partially digesting a plurality of pieces of cartilage, obtaining a piece of cartilage from the subject and mincing the piece of cartilage to form the plurality of pieces of cartilage.

19. The method of claim 15, the method further comprising, prior to inserting the partially digested cartilage into a body of a subject, encapsulating the partially digested cartilage in a hydrogel.

20. The method of claim 18, the method further comprising, prior to inserting the partially digested cartilage into a body of a subject, incorporating the encapsulated partially digested cartilage into a scaffold, and wherein inserting the partially digested cartilage into a body of a subject proximate the chondral defect comprises inserting the scaffold into a body of a subject proximate the chondral defect.
